(19) United States
(12) Patent Application Publication
(10) Pub. No.: US 2006/0078515 A1
(43) Pub. Date: Apr. 13, 2006

(54) Sunscreen wipes

(76) Inventor: Lori Kamrin-Balfour, Seattle, WA (US)

Correspondence Address: BLACK LOWE & GRAHAM, PLLC, 701 FIFTH AVENUE, SUITE 4800, SEATTLE, WA, 98104, US

(21) Appl. No.: 10/965,081

(22) Filed: Oct. 13, 2004

(51) Int. Cl. A61K 8/00; A61K 9/70

(52) U.S. Cl. 424/59; 424/443

(57) ABSTRACT

The invention relates to a product comprising a wet wipe and a sunscreen (colored or white) emulsion. The sunscreen emulsion is impregnated into the wet wipe resulting in the even spread of the colored sunscreen with more convenience, and significantly reducing the mess and waste normally resulting from the application of emulsions by hand.

### FIELD OF THE INVENTION

This invention relates generally to sunscreen application products, including wipes for the application of sunscreen.

### BACKGROUND OF THE INVENTION

Sunscreens are substances or compositions applied to the skin to protect the skin from sunburn caused by the sun's ultraviolet rays. When uniformly applied to the body, sunscreens can be highly effective in protecting against sunburn. However, sunscreen failure can occur when areas of the body are missed because the sunscreen is hard to see or visualize after being applied or rubbed onto the skin. Children are at greater risk of sunburn than adults, since coverage on children's skin is more likely to be incomplete, uneven or inconsistent. A color indicator has been added to some sunscreens, making it visibly noticeable when being applied to the skin. The coloration substantially disappears when the sunscreen emulation dries after it is spread on the skin and is rubbed out. Examples of such colored sunscreens are described in U.S. Pat. Nos. 6,290,936; 6,146,618; and 6,099,825, which are incorporated by reference.

Though the colored tints are helpful in providing full distribution of sunscreen, the do not address the mess associated with the application. Because of the substantial mess involved in applying sunscreen, a need arises for another method of dispensing the sunscreen emulsion. Ideally, an improved application method can also incorporate tints to allow even distribution.

### SUMMARY OF THE INVENTION

In its preferred form, the present invention comprises a colored sunscreen wipe. The wipe is impregnated with colored sunscreen to allow an ideal amount of sunscreen to be dispersed over the skin, at the same time leaving the hands of the applier free of excess sunscreen. The need for colored sunscreen wipes arises especially when traveling, or at the beach or pool, and when access to a source of water for cleaning the hands after application is limited. The wipe also allows only the necessary amount of sunscreen to be applied evenly to the skin, thus eliminating wasteful amounts of sunscreen in the application process.

### BRIEF DESCRIPTION OF THE DRAWINGS

The preferred and alternative embodiments of the present invention are described in detail below with reference to the following drawings.

FIG. 1 is a top plan view of a preferred wipe;

FIG. 2 is an end view of a preferred wipe containing sunscreen;

FIG. 3 is a perspective view of a preferred wipe container;

FIG. 4 is a perspective view of an alternate preferred wipe container;

FIG. 5 is a side view of a preferred method for folding and interleaving wipes in a container; and

FIG. 6 is a flow diagram for a preferred method of using a sunscreen wipe.

### DETAILED DESCRIPTION OF THE PREFERRED EMBODIMENT

The preferred embodiment of the present invention relates to a swatch of fibrous materials incorporating a desired amount of sunscreen, ideally with a colored tint. The wipes reduce the amount of wasted sunscreen emulsion applied to the skin and facilitate even coverage to protect the skin from ultraviolet radiation. According to one embodiment, the wipes are folded and arranged in a stacked configuration inside a suitable container for consumer sale.

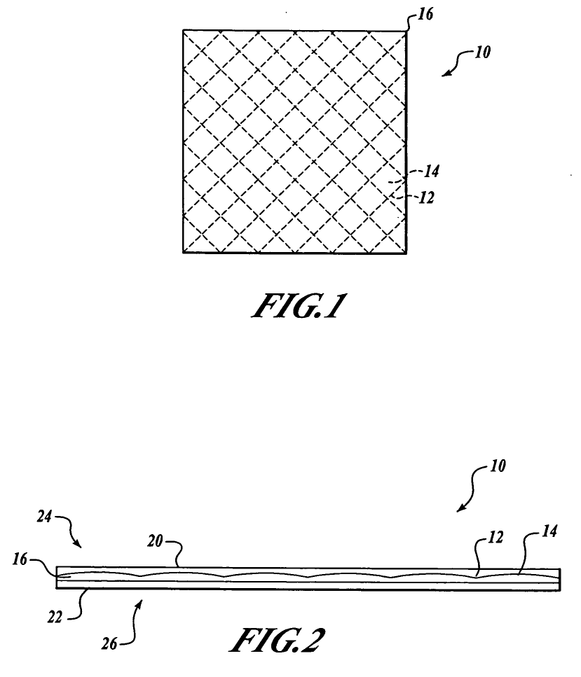

FIG. 1 shows an exemplary preferred wipe 10, comprising a swatch of fibrous material 16. The swatch can be formed from cotton or other materials commonly associated with “cloth,” or may alternatively be formed from paper or pulp-based materials commonly associated with paper towels and baby wipes. Likewise, the swatch may be formed from a blend of various materials, so long as it remains relatively flexible. Ideally, the swatch is capable of absorbing and holding a sufficient amount of sunscreen. In certain embodiments, however, the sunscreen is principally not absorbed into the swatch, but rather remains substantially atop the swatch.

As indicated in FIG. 1, the swatch preferably is “quilted,” meaning that it includes a plurality of generally raised regions 14 and depressed regions 12. The quilting enables the swatch to hold a greater amount of sunscreen in order to cover a larger area.

FIG. 2 illustrates an end view of the wipe depicted in FIG. 1. Thus, the wipe of FIG. 2 comprises a swatch 16 having a plurality of raised regions 14 and depressed regions 12. A layer of sunscreen 20 is shown on the top portion of the wipe, with a relatively greater amount of sunscreen retained within the depressed regions. In some embodiments, the top and bottom of the wipe will be substantially symmetrical, with a layer of sunscreen on either side. In alternate embodiments, however, an additional material is applied to or incorporated into the swatch to inhibit the sunscreen from migrating from the top surface 24 to the bottom surface 26. Thus, in one embodiment a generally sunscreen-impenetrable layer 22 is provided on the bottom surface 26 so that the bottom surface of the wipe remains dry. The inhibiting layer may be formed from plastic, rubber, fluoropolymers, nylon, or other materials. In alternate embodiments, a similar layer is woven into the wipe, or the wipe is formed from less absorbent materials such that the layer of sunscreen on top surface 24 of the swatch is inhibited from flowing toward the bottom surface 26.

The wipes contain a sunscreen emulsion solution which is absorbed into or rests on top of one side of the wipes. The sunscreen may be a standard white color or, in some embodiments, may contain a colored tint. The amount of the sunscreen solution contained in each wet wipe may vary depending upon the type and composition of the sunscreen (e.g. waterproof, SPF 2, SPF 50, etc.).

The sunscreen emulsion that is impregnated into the wipe can contain a water-soluble color dye (color indicator) in an amount sufficient to enable the sunscreen to be readily visualized (i.e. colored) when initially applied to the skin, such that when the sunscreen emulsion dries after being spread on the skin or is rubbed into the skin using one's hand or fingers, the color substantially disappears. One or more water-soluble dyes can be employed in the composition, preferably in an amount ranging from about 0.0005 to about 0.5% by weight of the sunscreen composition. A suitable water-soluble color dye is a blend of Ext. DC violet #2(95%) and Ext. DC red #3 (5%).

The sunscreen compositions can also contain a sun screening effective amount of one or more oil-soluble or water-soluble sun screening UV_A and UV_B actives. Water is employed in the sunscreen in amounts effective to form the emulsion and solubilize the ingredients. A waterproofing agent may also be added to provide waterproofing characteristics to the emulsion. Suitable waterproofing agents include copolymers derived from polymerization of octadecene-1 and malefic anhydride.

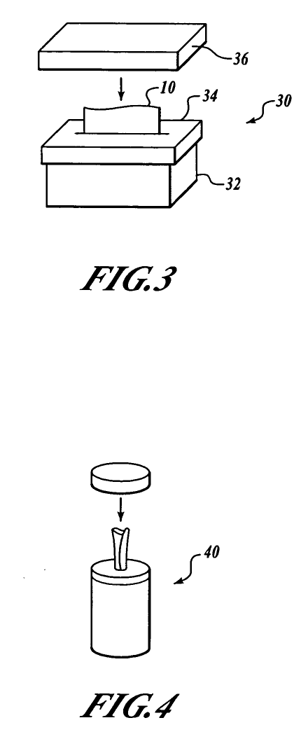

The colored (or uncolored) sunscreen wipes may be dispensed in any number of boxes or bags, such as those shown in FIGS. 3 and 4. Thus, as one example, a dispenser box 30 may be used, including a container body 32 and dispenser lid 34 having a slot to enable a wipe 10 to be removed through the dispenser lid 34. A substantially air-tight, resealable lid 36 is also provided to allow the wipes to stay saturated with the colored sunscreen. In the form of FIG. 4, a similar dispenser package is provided in a cylindrical shape. Either of the packaging types of FIGS. 3 and 4 may be used without the dispenser lids.

In yet another embodiment, one or more wipes is provided in individually-wrapped packages. In this form, a single wrap is folded multiple times to form a pocket-sized square (or other shape), then sealed in a water-tight and air-tight package that can be opened for individual use. Such a package may also include two or more wipes in a single package.

When using a dispenser package with wipes or facial tissues, it is common to fold the wipe or tissues in an overlapping arrangement in which pulling one wipe from the package will urge an adjacent wipe out of the package with it. In one embodiment, such as that shown in FIG. 3, a similar folding arrangement is used for the sunscreen wipes.

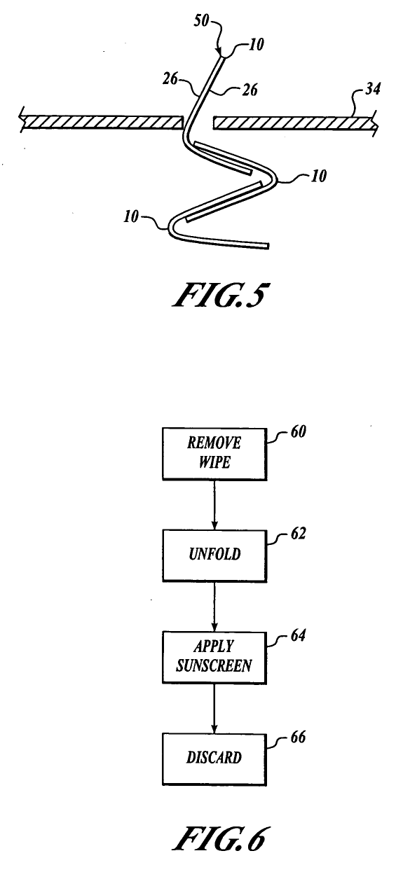

In an embodiment in which the wipes contain sunscreen on a top surface 24 and have a substantially dry bottom surface 26, it is useful to store the wipes within a container such that the sunscreen on the top surface of a wipe does not contaminate the dry and clean bottom surfaces of other wipes. A preferred arrangement for accomplishing this result is to fold the wipes before interleaving them within the container. Thus, as shown in FIG. 5, a plurality of wipes 10 are folded generally in half so that the dry bottom surface 26 faces outward and the wet sunscreen-containing top surface faces inward. Only the bottom surfaces of adjacent wipes contact one another, thereby ensuring that each wipe is removed from the container with one wet side and one dry side. In addition, if the wipes are arranged within the container such that the folded end 50 emerges from the container before the open end, it will better ensure that the sunscreen does not dry out, even if the top lid 36 is removed or not used.

As shown in FIG. 5, a C-folded arrangement is used for interleaving adjacent wipes. Certainly a Z-folded or other arrangement may be used to enable a continuous flow of wipes to emerge from the package, one at a time.

In an alternative configuration, the wet wipes may take the form of continuous webs of material which include perforations to separate the individual wet wipes and which are wound into rolls and packaged in plastic containers. An example of such a container is shown in FIG. 4, and described more fully in U.S. Pat. Nos. 6,613,729 and 6,696,070.

A preferred method for using the sunscreen wipe is shown in FIG. 6. Initially, a wipe is removed from a package containing one or more wipes in a first step 60. The wipe is unfolded 62 if necessary (depending on the packaging method) to expose the sunscreen-containing top surface. In the embodiment having a substantially dry side and a substantially wet side, the dry side is placed against the palm of the hand with the wet side exposed. This allows the sunscreen to be applied 64 by rubbing the wipe against the skin wherever sunscreen is desired. If the sunscreen includes a colored tint, the user will readily be able to determine whether the coverage has been even. Finally, when the sunscreen is sufficiently, applied, the wipe is discarded 66.

The colored sunscreen wipe will allow the consumer to use the sunscreen emulsion within a convenient wet wipe, thus achieving maximum possible uniformity of application to the skin with the least amount of mess possible.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A sunscreen wipe comprising:
a fibrous swatch having a top surface and a bottom surface; and
a layer of sunscreen emulsion adjacent the top surface.

2. The wipe of claim 1, wherein the sunscreen further comprises a dye.

3. The wipe of claim 2, wherein the dye is configured such that it is visible when the sunscreen is initially applied to the skin, but fades as it dries.

4. The wipe of claim 3, wherein the swatch is quilted.

5. The wipe of claim 3, wherein the top surface of the swatch further comprises raised regions and depressed regions.

6. The wipe of claim 3, wherein the swatch further comprises a means for inhibiting the sunscreen from migrating to the bottom surface.

7. The wipe of claim 3, wherein the swatch further comprises a substantially sunscreen impermeable layer, and further wherein the bottom surface is substantially devoid of sunscreen.

8. The wipe of claim 7, wherein the wipe is contained in a container with a plurality of additional wipes, each of the additional wipes being formed substantially the same as the wipe of claim 7.

9. The wipe of claim 8, wherein the wipe is folded to form an outer surface such that the outer surface is formed primarily by the bottom surface of the swatch.

10. The wipe of claim 8, wherein each of the plurality of wipes is folded and interleaved such that the bottom surfaces of adjacent wipes are in contact with one another.
